(19) United States
(12) Patent Application Publication
(10) Pub. No.: US 2014/0016747 A1
(43) Pub. Date: Jan. 16, 2014

(54) RADIATION IMAGING APPARATUS

(71) Applicant: CANON KABUSHIKI KAISHA, Tokyo (JP)

(72) Inventor: Tetsuo Watanabe, Utsunomiya-shi (JP)

(21) Appl. No.: 13/936,665

(22) Filed: Jul. 8, 2013

(30) Foreign Application Priority Data
Jul. 13, 2012 (JP) 2012-157966

(51) Int. Cl. A61B 6/00

(52) U.S. Cl. CPC A61B 6/56
USPC 378/62; 378/98

(57) ABSTRACT

A mobile radiation imaging apparatus comprising: a generation unit configured to emit radiation; a positioning unit configured to support the generation unit; a moveable cart configured to moveably hold the positioning unit; a storing unit for a radiation imaging unit that includes a radiation sensor and a first battery, the storing unit having a terminal that supplies power to the radiation imaging unit; a second battery configured to connect to the terminal, the generation unit, and a cable for receiving a supply of power from an external power source; and a control unit configured to control power supply from the second battery to the first battery based on a remaining amount of the first battery and a remaining amount of the second battery.

### BACKGROUND OF THE INVENTION

1. Field of the Invention

The present invention relates to a radiation imaging apparatus.

2. Description of the Related Art

Conventionally, apparatuses that irradiate a target object with X-rays and capture an X-ray image by detecting the intensity distribution of the X-rays that pass through the target object have been widely used in the fields of industrial non-destructive inspections and medical diagnoses. Film/screen formats and CR formats are examples of common methods of this kind of radiation imaging. With these methods, photosensitive film or a fluorescent plate that accumulates an image as a latent image is inserted into a JIS Z 4905 standardized storing case called a cassette and is used for imaging. On the other hand, formats in which high-quality radiation images are acquired by converting radiation images into electrical signals and reproducing them as visible images on a CRT or the like after carrying out image processing on the electrical signals have become widespread due to recent advancements in digital technology.

FIG. 8 shows an example of a configuration of a conventional radiation imaging system. A radiation detection sensor 104 is built into a radiation imaging apparatus 103. A radiation generation apparatus 101 irradiates a subject 102 with radiation. The radiation that passes through the subject 102 is converted into visible light via fluorescent material by the radiation detection sensor 104, and is detected as electrical signals by photoelectric conversion elements arranged in a two-dimensional lattice. A control unit 105 performs control of readout driving, image transfer, and the like with respect to the radiation detection sensor 104, performs digital image processing on images output from the radiation detection sensor 104, and displays radiation images of the subject 102 on a monitor 106. In such a radiation imaging system, a detection panel is installed on a dedicated mount depending on the imaging configuration, such as a standing or recumbent configuration, and is used as needed.

Conventionally, radiation imaging systems installed in specified radiation rooms have been used. However, in recent years, in order to enable more rapid and wider-range imaging of sites, a thin, lightweight, transportable imaging apparatus (electronic cassette) has been developed.

Japanese Patent Laid-Open No. 11-99144 provides a mobile radiation imaging apparatus that is applied not only to cassette imaging in a radiation room, but also to the field of doctors' round visits.

However, the mobile radiation imaging apparatus disclosed in Japanese Patent Laid-Open No. 11-99144 needs at least enough power to generate radiation, and moreover, the needed energy is furthermore increased due to the imaging apparatus being combined with an electronic cassette. The power that can be supplied depends on the capacity of a battery, and if the capacity thereof is increased, the weight and size increase, which also affects the size of the apparatus itself. Conversely, if the capacity is suppressed to a low amount, the number of instances of charging increases and the operation availability decreases. Accordingly, in order to perform imaging efficiently, it is necessary to manage power consumption giving consideration to the balance between power used for radiation generation and power used by the imaging apparatus.

Additionally, in recent years, the implementation of wireless communication in electronic cassettes has progressed, and has been widely used due to the benefit of being able to operate in a cable-less state. In such a case, when a battery that is to be the power source of an electronic cassette is loaded in a mobile radiation imaging apparatus, there is a possibility that vibrations during movement will subject the power charge terminal area to stress, compromising the reliability thereof.

In view of the aforementioned problem, an object of the present invention is to provide a technique that manages effective power consumption giving consideration to the balance between power for radiation generation and power for the imaging apparatus. Also, the present invention provides a technique that reduces malfunction by suppressing stress caused by vibration that the power charge terminal area is subjected to.

### SUMMARY OF THE INVENTION

According to one aspect of the present invention, there is provided a mobile radiation imaging apparatus comprising: a generation unit configured to emit radiation; a positioning unit configured to support the generation unit; a moveable cart configured to moveably hold the positioning unit; a storing unit for a radiation imaging unit that includes a radiation sensor and a first battery, the storing unit having a terminal that supplies power to the radiation imaging unit; a second battery configured to connect to the terminal, the generation unit, and a cable for receiving a supply of power from an external power source; and a control unit configured to control power supply from the second battery to the first battery based on a remaining amount of the first battery and a remaining amount of the second battery.

Further features of the present invention will be apparent from the following description of exemplary embodiments with reference to the attached drawings.

### BRIEF DESCRIPTION OF THE DRAWINGS

FIG. 1 is a cross-sectional side view of an imaging unit 10 according to a first embodiment.

FIG. 2 is a diagram for describing a configuration of an X-ray imaging apparatus 20 according to the first embodiment.

FIGS. 3A-3B are flowcharts showing a procedure of processing carried out by the X-ray imaging apparatus 20 according to the first embodiment.

FIG. 4 is a cross-sectional side view of an imaging unit 50 according to a second embodiment.

FIG. 5 is a diagram for describing a configuration of an X-ray imaging apparatus 60 according to the second embodiment.

FIG. 6 is a diagram for describing a configuration of an X-ray imaging apparatus 90 according to a third embodiment.

FIG. 7 is a cross-sectional view for describing a state of an imaging unit 10 stored in a storing unit 40 according to the third embodiment.

FIG. 8 is a diagram showing an example of a configuration of a conventional radiation imaging system.

### DESCRIPTION OF THE EMBODIMENTS

Exemplary embodiments of the present invention will now be described in detail with reference to the drawings. It should be noted that the relative arrangement of the components, the numerical expressions and numerical values set forth in these embodiments do not limit the scope of the present invention unless it is specifically stated otherwise.

### First Embodiment

A medical X-ray imaging apparatus that captures an image of a test subject with use of X-rays will be described below as an example of a radiation imaging apparatus, but the present invention can also be applied to an X-ray imaging apparatus that captures an image of another subject, or a radiation imaging apparatus that uses another type of radiation.

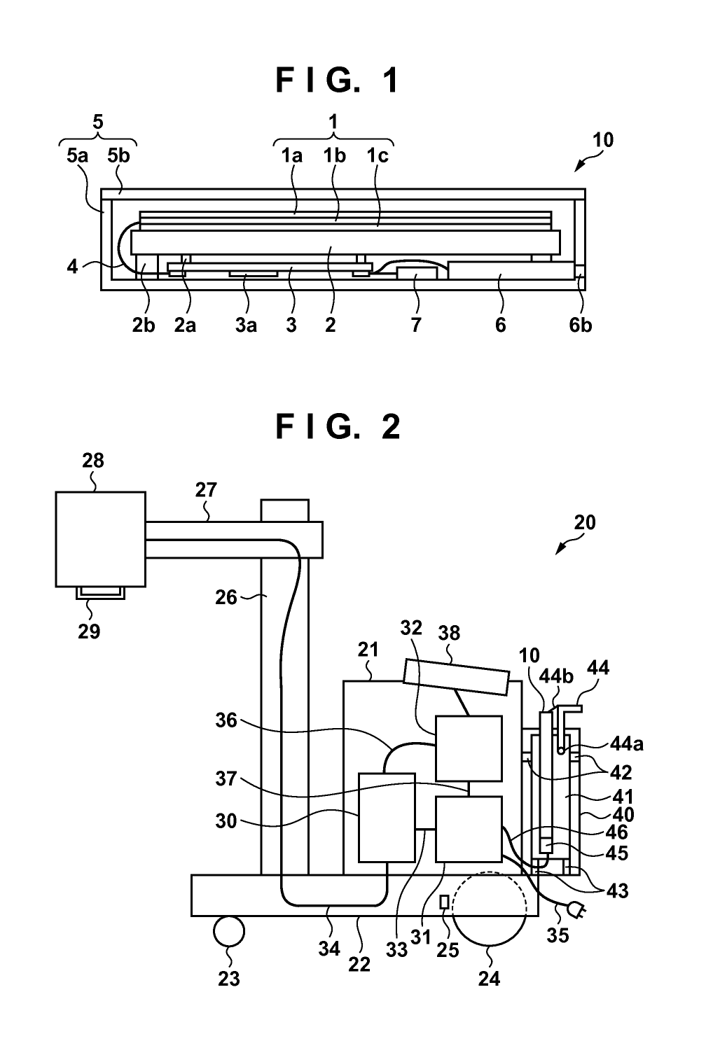

FIG. 1 is a cross-sectional side view of an imaging unit 10 according to the first embodiment. The imaging unit 10 is an electronic cassette that includes an X-ray image detection panel 1, a base 2, a circuit board 3, a flexible circuit board 4, a housing 5, a battery 6 (first power source unit), and a wireless communication unit 7.

The X-ray image detection panel 1 basically has a fluorescent screen 1a, photoelectric conversion elements 1b, and a substrate 1c. A glass plate is often used as the substrate 1c, due to the need for there to be no chemical action with the semiconductor element, the ability to withstand the temperature in the semiconductor process, and dimensional stability. On this substrate 1c, the photoelectric elements 1b are formed in a two-dimensional array by a semiconductor process. A resin board coated with a fluorescent material made of a metallic compound is used as the fluorescent screen 1a, and is integrated with the substrate 1c by means of adhesion.

The metallic base 2 has a protrusion 2a and a support unit 2b, and fixedly supports the X-ray image detection panel 1. In this way, mechanical strength is ensured. The circuit board 3 includes an electronic component 3a that processes electrical signals obtained by conversion by the photoelectric conversion element 1b. The flexible circuit board 4 is a flexible substrate, and can curve as shown in FIG. 1. The housing 5 can store various substrates and the like in its interior and it includes a housing body 5a and a housing cover 5b. The battery 6 is a chargeable power source that supplies power to the circuit board 3 and the like in order to drive the imaging unit 10. An electrical contact terminal 6b for charging the battery 6 is included in the imaging unit body 5a. The wireless communication unit 7 wirelessly communicates with an external device and performs the transmission of signals such as image signals, control signals.

The circuit board 3 is connected to the photoelectric conversion elements 1b by the flexible circuit board 4, and is fixed to the protrusion 2a that is provided on the back side of the base 2. Also, the base 2 is fixed to the housing body 5a via the support unit 2b, and is sealed by the housing cover 5b that transmits X-rays. Thus, the imaging unit 10 is configured. This imaging unit 10 is combined with a mobile X-ray generation apparatus for doctors' round visits and is used as a mobile X-ray imaging apparatus, which will be described later.

An example of a configuration of a mobile X-ray imaging apparatus 20 according to the first embodiment will be described below with reference to FIG. 2.

Multiple wheels 23 and 24 for configuring a traveling cart are included on a bottom portion 22 of a main body portion 21 of the X-ray imaging apparatus 20, and the position of the entire X-ray imaging apparatus 20 can be moved arbitrarily. A movement detection unit 25, which detects rotation of the wheel 24, has a function of detecting a moving state of the X-ray imaging apparatus 20. This can be realized with a simple method such as a rotary encoder. On the other hand, included in front of the main body portion 21 (left side of the page) are a vertical support pillar 26 that is supported so as to be rotatable around a shaft, and an arm 27 that extends horizontally with respect to the support pillar 26 and is supported so as to be movable in a perpendicular direction.

An X-ray generation unit 28 that includes an X-ray tube is attached at the tip of the arm 27 such that it can move in a horizontal direction along the arm 27 and its radiation direction can be adjusted arbitrarily. A touch sensor is provided on a grip 29 that is provided on the X-ray generation unit 28, and when the grip 29 is squeezed, the movement of various mechanisms such as the arm 27 and the X-ray generation unit 28 is unlocked, and the X-ray tube can be positioned in a desired orientation.

Also, disposed within the main body portion 21 are an electrical circuit unit 30 that controls tube driving for X-ray irradiation and the locking of mechanism units and the like, a battery 31 (second power source unit), and a control unit 32 for controlling the imaging unit 10 and the X-ray generation unit 28 and for controlling the entire X-ray imaging apparatus 20. The control unit 32 has a communication unit for communicating with the imaging unit 10, a storage unit that stores information such as images, an input/output unit, an interface unit that coordinates the X-ray generation unit 28 and the imaging unit 10, a power source output control unit, and a controller for controlling all of these (not shown).

Power for X-ray irradiation is supplied by a first power supply system to which power is supplied from the battery 31, and the control unit 32 can control the switching on and off of power. The first power supply system has the battery 31, the electrical circuit unit 30 of the X-ray generation unit 28, wiring 33 that connects them both, and wiring 34 that is connected to the X-ray generation unit 28.

On the other hand, the storing unit 40 for storing the imaging unit 10 is provided in the main body portion 21, it stores the imaging unit 10 when the apparatus is moving, and when the imaging unit 10 is to be used, it is taken out from the storing unit 40 and used. A charger 41 for charging the battery 6 built into the imaging unit 10 is provided inside the storing unit 40, and is supported with respect to the vertical and horizontal directions by multiple anti-vibration members 42 and 43, such as springs or cushions. The imaging unit 10 is held with a latch member 44 such that its relative position with respect to the charger 41 is restricted. The latch member 44 is supported to the main body of the charger 41 so as to be able to rotate around a shaft 44a, and the imaging unit 10 is restricted by a claw 44b provided at its tip. When the imaging unit 10 is to be taken out, it can be taken out by performing an operation for rotating on the latch member 44 in the direction of canceling restriction. Due to having such a structure, the relative positions of the terminals of the imaging unit 10 and the charger 41 can be kept stable and a stable charging state can be maintained, even during movement.

Also, the charger 41 has a connection unit 45 for supplying power to the imaging unit 10, is connected to the battery 31 built into the main body portion 21 of the X-ray imaging apparatus 20 via wiring 46, and configures the second power supply system for supplying power to the battery 6 that is inside the imaging unit 10. The switching on and off of the output of this second power supply system is controlled by the control unit 32 that is connected by wiring 37. The battery 31 is connected to the electrical circuit unit 30, the charger 41 for the imaging unit 10 and the control unit 32, and supplies power to these, and is connected to an AC cable 35 for charging the battery 31 using an external source. The battery 31 is charged in advance with power for running the X-ray imaging apparatus 20, via the AC cable 35 in a period of time in which imaging is not being performed. The AC cable 35 may be configured so as to be storable inside the main body portion 21 when the apparatus is moving.

An input/output unit 38, which is used for performing operations and the like on the X-ray imaging apparatus 20 includes a monitor for display output and an input device, and is disposed on the upper portion of the main body portion 21. Conceivable examples of input devices include a selection key button that switches the selection position on the monitor, a touch panel, and the like. On the monitor, the imaging site is selected and the imaging unit 10 transitions to a state in which imaging is enabled, an operation menu is displayed through which imaging conditions such as the tube voltage, tube current, and irradiation duration of the X-ray generation unit 28 are set, and imaging is performed by selecting an item via the input/output unit 38. Also, processes such as trimming and rotation are performed on the captured image, and a series of operations up to and including the saving of the captured image in the storage unit built into the control unit 32 are performed with use of the input/output unit 38. Also, the control unit 32 manages the information on the patient for whom the order was made, the imaging conditions, and the imaging history, and an operator can check lists of patient information and the like from the input/output unit 38. This information is transferred by communicating with an in-hospital external terminal via a unit that communicates with an external device and is connected to the control unit 32.

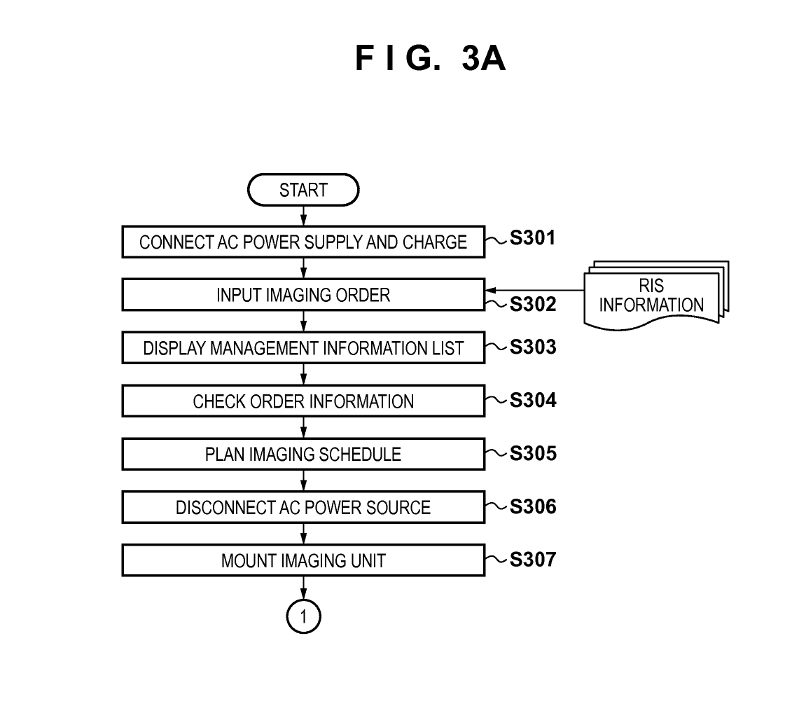

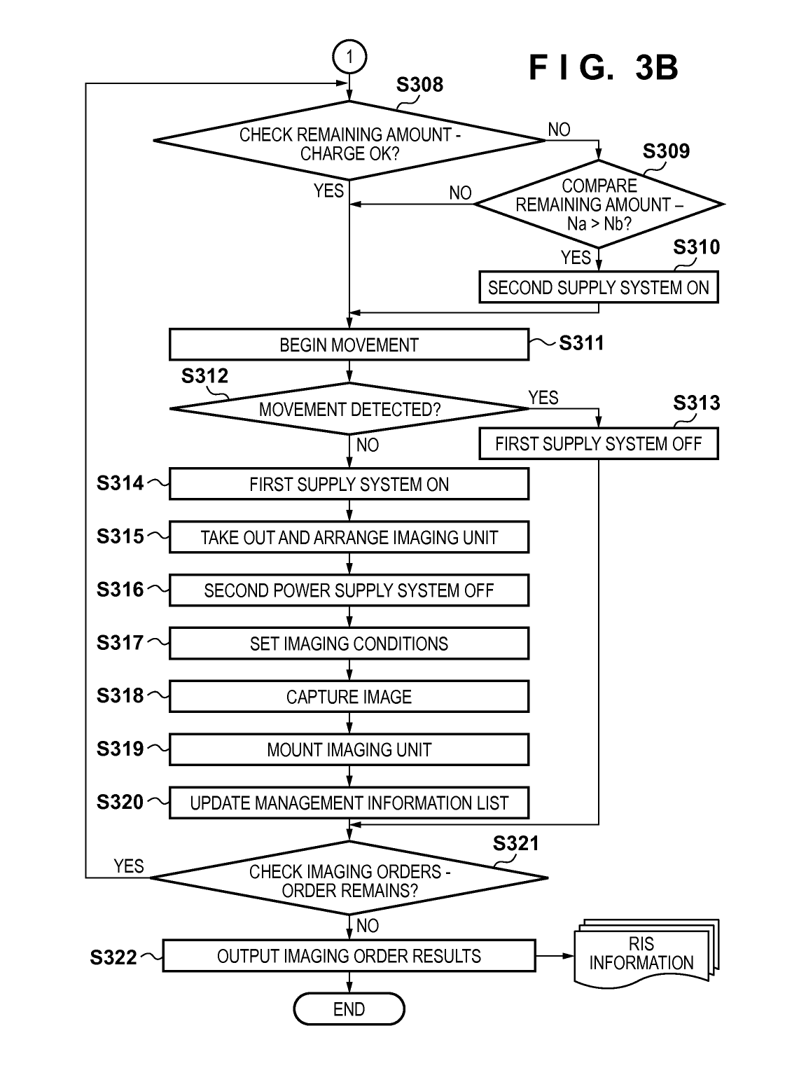

Next, a procedure of processing performed by the X-ray imaging apparatus 20 according to the first embodiment will be described with reference to FIGS. 3A-3B.

In step S301, as a preparatory state, the technician of the X-ray imaging apparatus 20 connects the AC cable 35 to a commercial power source in order to activate the X-ray imaging apparatus 20, and charges the battery 31. Note that this processing may be executed in advance.

In step S302, the X-ray imaging apparatus 20 acquires order information from an in-hospital RIS terminal via a unit that communicates with an external device (not shown) and uploads it to the control unit 32. In step S303, the input/output unit 38 displays information acquired from the RIS terminal as a list.

In step S304, the technician checks the information displayed on the input/output unit 38. The content that is displayed is patient information such as the patient's name and gender, a hospital room number, imaging conditions such as the imaging site and imaging orientation, and the like.

In step S305, the technician plans the approximate schedule of the diagnostic imaging giving consideration to priority level and movement route, based on the information displayed on the input/output unit 38. In step S306, when preparations end, the technician disconnects the AC cable 35 from the commercial power source and stores it in the main body.

In step S307, the technician mounts the imaging unit 10 to the X-ray imaging apparatus 20. The imaging unit 10 is mounted on the charger 41, which is built into the storing unit 40 that is provided in the main body. At this time, the battery 6 of the imaging unit 10 is connected to the second power supply system via the connection unit 45 of the charger 41.

Also, in the process leading to this state, as described above, the claw 44b of the latch member 44 comes into contact with the imaging unit 10, the imaging unit 10 is held such that relative movement with respect to the charger 41 is restricted, and the imaging unit 10 along with the charger 41 are supported to the main body such that vibration is prevented.

In step S308, the control unit 32 checks the remaining amount of the battery of the imaging unit 10. If it is determined that the remaining battery amount is greater than or equal to a pre-set remaining amount, and charging is sufficient (S308: YES), the processing moves to step S311. On the other hand, if it is determined that the remaining battery amount is less than the pre-set remaining amount, and charging is insufficient (S308: NO), the processing moves to step S309.

In step S309, the control unit 32 compares the remaining amount that can be supplied to the X-ray generation unit 28, and the remaining amount of the battery of the imaging unit 10. Specifically, the remaining number of possible image captures is estimated based on the remaining amounts, and it is determined whether or not a remaining number of possible image captures Na of the X-ray generation unit (second number of possible image captures) is greater than the number of possible image captures of the imaging unit 10 (first number of possible image captures). If it is determined that Na is greater than Nb (S309: YES), the processing moves to step S310. On the other hand, if it is determined that Na is less than or equal to Nb (S309; NO), the processing moves to step S311.

In step S310, the control unit 32 switches on the supply of power from the second power supply system to the battery 6 in the imaging unit 10. In this way, the number of captures than can be performed in imaging can be optimally managed by correcting the balance of power consumption by both the X-ray generation unit 28 and the imaging unit 10 and controlling the supply to the power supply systems.

In step S311, when preparation is finished, the technician begins movement to a predetermined hospital room by pushing the X-ray imaging apparatus 20 in accordance with the planned schedule. In step S312, the control unit 32 detects the movement speed of the X-ray imaging apparatus 20 based on detection results from the movement detection unit 25 that detects rotation of the wheel 24. If the movement speed of the X-ray imaging apparatus 20 is at or above a predetermined speed, a moving state is detected. If the moving state is detected (S312: YES), the processing moves to step S313. On the other hand, if the moving state is not detected, that is to say, if a stopped state is detected (S312: NO), the processing moves to step S314.

In step S313, the control unit 32 switches off the supply of power from the first power supply system to the X-ray generation unit 28. At this time, the X-ray generation unit 28 is disposed out of the way such that the technician can safely move the X-ray imaging apparatus 20. On the other hand, even if vibration or impact occurs due to difference in level on the floor or the like, transfer thereof to the charger 41 and the imaging unit 10 is suppressed by the anti-vibration members 42 and 43, and the connection terminal portions can safely maintain a connected state, and therefore, reliability can be ensured.

In step S314, when the X-ray imaging apparatus 20 reaches the pre-determined hospital room and stops, the control unit 32 switches on the supply of power from the first power supply system to the X-ray generation unit 28. Thus, operation of the X-ray generation unit 28 is enabled. In step S315, the technician takes out the imaging unit 10 from the X-ray imaging apparatus 20 by operating the latch member 44 and canceling the restriction of the imaging unit 10, and disposes the imaging unit 10 at the imaging position.

In step S316, the control unit 32 switches off the supply of power from the second power supply system to the battery 6 in the imaging unit 10 in response to the imaging unit 10 being taken out. In step S317, the technician adjusts the position of the X-ray generation unit 28 to a predetermined imaging orientation, selects an imaging site on the monitor, and sets imaging conditions such as the tube voltage of the X-ray generation unit 28 and the mAS value.

After imaging conditions are set, in step S318, the control unit 32 executes X-ray imaging. After imaging ends, in step S319, the technician stores the imaging unit 10 in the storing unit 40. In step S320, the control unit 32 automatically updates the list so as to reflect the fact that imaging has ended as management information.

In step S321, the control unit 32 checks the imaging order, and determines whether or not an imaging order remains. If it is determined that an imaging order remains (S321: YES), the processing returns to step S308, the X-ray imaging apparatus is moved to the next hospital room, and the flow is repeated. On the other hand, if it is determined that no imaging order remains (S321; NO), the processing moves to step S322.

In step S322, the technician returns the X-ray imaging apparatus 20 to a predetermined waiting position. The control unit 32 simultaneously outputs imaging order processing results to the RIS and ends diagnostic imaging.

Also, as a variation, in order to suppress the total charge amount, control may be performed such that the first power supply system and the second power supply system do not enter a powered-on state simultaneously. In such a case, when the first power supply system is switched on in step S314 in the aforementioned flow, the second power supply system may be switched off at a timing that is synchronous with step S314, before the process of taking out the imaging unit 10 in step S315.

As described above, according to the present embodiment, efficient power consumption giving consideration to the balance between power for radiation generation and power for an imaging apparatus can be managed. Also, by suppressing stress caused by vibration that the charge terminal unit is subjected to, malfunctions can be suppressed, and reliability can be improved.

### Second Embodiment

In the first embodiment, a configuration was described in which the battery 6 that is built into the imaging unit 10 was charged directly. In contrast to this, in the second embodiment, a configuration will be described in which a battery than is detachable from an imaging unit is removed from the imaging unit and charged, or a battery that is different from the battery mounted in the imaging unit is charged.

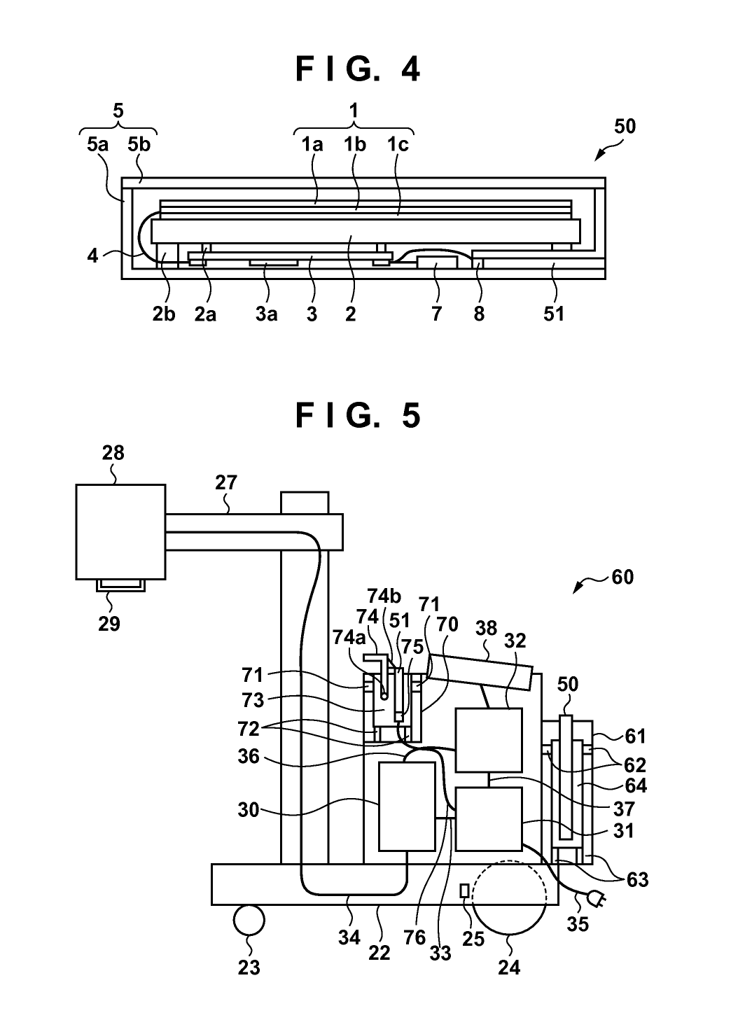

FIG. 4 is a cross-sectional side view of an imaging unit 50 according to the second embodiment. Also, FIG. 5 is a diagram showing a configuration of a mobile X-ray imaging apparatus 60 according to the second embodiment. Identical reference numerals are assigned to configurations that are shared by the first embodiment, and the descriptions thereof will be omitted.

In FIG. 4, the imaging unit 50 has a battery 51 that is detachable and exchangeable, unlike the first embodiment, and it is in a configuration in which the mounted battery element is charged. When mounted in the imaging unit 50, the battery 51 can supply power to the electrical substrate 3 and the like in the interior via a contact 8. A task of exchanging the battery is involved when the remaining amount of the battery 51 mounted in the imaging unit 50 is gone, but the fact that the battery can be promptly exchanged is a benefit.

In FIG. 5, the X-ray imaging apparatus 60 has a storing unit 61 that stores the imaging unit 50, and a power source storing unit 70 that stores the battery 51 when it is removed from the imaging unit 50. Inside the storing unit 61, a holder 64 that holds the imaging unit 50 is supported to the main body via anti-vibration members 62 and 63 such that vibration is prevented. On the other hand, a charger 73 is mounted inside the power source storing unit 70 of the battery 51. The battery 51 is held by a latch member 74 such that the relative movement of the battery 51 and the power source storing unit 70 is restricted in a state in which the terminal of the battery 51 and the terminal 75 of the charger 73 are connected. If the battery 51 is to be taken out of the power source storing unit 70, it can be taken out by rotating the latch member 74 around a shaft 74a, and separating a claw 74b that is provided at the tip from the battery 51, thus canceling restriction. Due to having such a structure, the relative positions of the terminals of the battery and the charger can be kept stable and a stable charging state can be maintained, even during movement.

Also, the charger 73 configures a second power supply system for connecting and supplying power to the battery 51, and is connected to the control unit 32, thus enabling control of the switching on/off of the output. The charger 73 communicates and supplies power via the wiring 76 so that charge information is transferred to the control unit 32, and so that control signals from the control unit 32 are received.

The imaging unit 50 periodically transfers remaining amount information of the mounted battery 51 to the control unit 32 using a wireless communication unit. The charge information of the battery 51 being charged is also transferred simultaneously from the charger 73 to the control unit 32. The control unit 32 compares the remaining amounts of the main body battery 31, the battery 51 that is mounted in the imaging unit 50, and another battery 51 that is mounted on the charger 73. The number of possible image captures Na that the X-ray generation unit 28 is able to perform is estimated from the remaining amount of the main body battery 31, and a total number of possible image captures Nc is estimated from the sum of the remaining amount of the battery 51 mounted on the imaging unit 50 and the remaining amount of the other battery 51 mounted on the charger 73.

Then, Na and Nc are compared, and if Na>Nc and furthermore the other battery 51 mounted on the charger 73 is not fully charged, the second power supply system is switched on to connect and supply power to the battery 51. On the other hand, if the other battery 51 mounted on the charger 73 is fully charged, or if Na≦Nc, the second power supply system is switched off.

The control unit 32 displays the remaining amount of the battery 51 mounted in the imaging unit 50, as well as the remaining amount of the other battery 51 mounted on the charger 73 on the input/output unit 38. By checking the state of the batteries on the input/output unit 38, the operator can switch the batteries at an appropriate time. Also, if Na≦Nc, the charge state of the overall apparatus can be understood by displaying that fact on the input/output unit 38. Alternatively, the level of the charge state may be announced using a sound.

As described above, according to the present embodiment, efficient power consumption giving consideration to the balance between power for radiation generation and power for an imaging apparatus can be managed. Also, by suppressing stress caused by vibration that the charge terminal unit is subjected to, malfunctions can be suppressed, and reliability can be improved.

### Third Embodiment

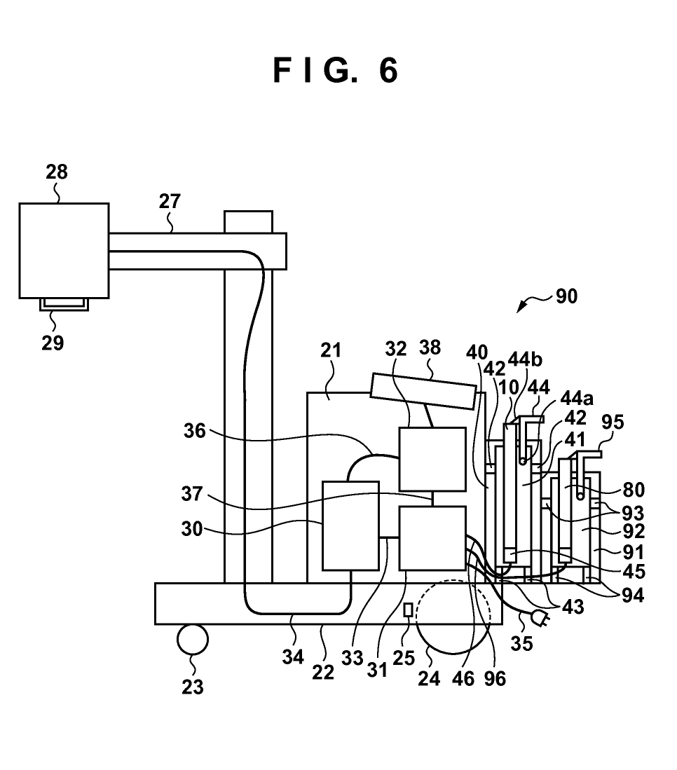

In the first embodiment, a configuration was described in which only one imaging unit 10 was mounted. In contrast to this, in the third embodiment, a configuration will be described in which multiple imaging units are mounted. FIG. 6 is a diagram showing a configuration of a mobile X-ray imaging apparatus 90 according to the third embodiment. Identical reference numerals are assigned to configurations that are shared by the first embodiment, and the descriptions thereof will be omitted.

In FIG. 6, the X-ray imaging apparatus 90 can have an imaging unit 10 and an imaging unit 80 that are of different sizes mounted thereto, and includes multiple corresponding chargers 41 and 92. The chargers include anti-vibration members 42, 43, 93, and 94, and latch members 44 and 95 for the imaging units, similarly to the first embodiment. For the different sizes, the combinations of half-cut size/big quarter-cut size and half-cut size/quarter-cut size are generally effective. By sorting and setting in advance the image captures best suited to the sizes, it can be understood which imaging unit will be used for each imaging order. Due to the charge states of batteries built into the imaging units being transferred to the control unit 32, the control unit 32 compares the imaging order and the battery charge states, and power supply control is performed giving priority to batteries whose remaining amounts are not full. Also, if the size of an imaging unit can be changed depending on the imaging order, order information may be switched. The total number of possible image captures Nd of the imaging units, and the number of possible image captures Na of the X-ray generation unit 28 are compared, and if Na>Nd and furthermore batteries mounted on chargers are not fully charged, the power supply system for supplying power to the battery of the imaging unit that is connected using the wiring 46 or the wiring 96 is switched on. If the batteries mounted on the chargers are fully charged, or Na≦Nd, the power supply system to the batteries mounted on the chargers is switched off. Thus, the number of captures that can be performed in imaging can be optimized while the balance of power consumption is corrected, by controlling the power supply from the chargers to the batteries of the imaging units.

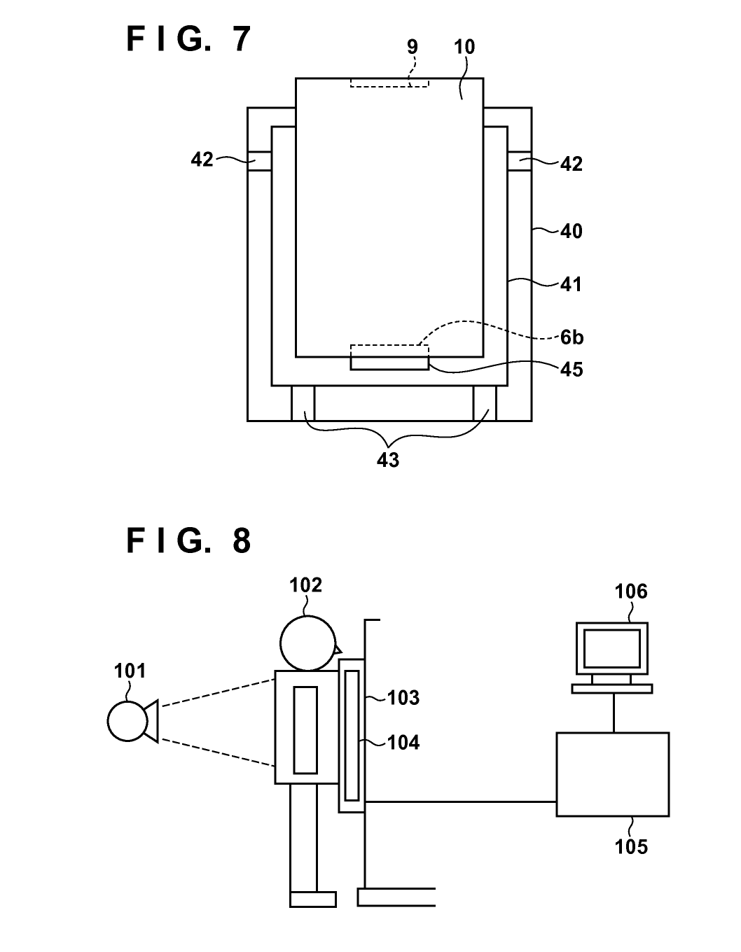

Next, FIG. 7 is an image showing the state of the imaging unit 10 when stored in the storing unit 40, and FIG. 7 is a cross-sectional view of FIG. 6 viewed from the side. A charger terminal 45 is provided in the charger 41 that is supported by the anti-vibration members 42 and 43. If the imaging unit 10 is to be mounted on the charger 41, the contact terminal 6b side is inserted facing the bottom side thereof. The display unit 9 is formed on the surface on the side opposite to the contact terminal 6b in the imaging unit 10. The display unit 9 displays the status of the imaging unit 10, and displays the remaining amount of the batteries within. In this way, both a stable connection state and a display that is easy to check are enabled by forming a display unit on an upward-facing surface that is exposed from the storing unit, and forming a contact terminal on the surface of the opposite side. The imaging unit 80 may also be configured similarly to the imaging unit 10.

As described above, according to the present embodiment, efficient power consumption giving consideration to the balance between power for radiation generation and power for the imaging apparatus can be managed. Also, malfunctions can be suppressed by suppressing stress caused by vibration that the charge terminal unit is subjected to, and reliability can be improved.

### Other Embodiments

Aspects of the present invention can also be realized by a computer of a system or apparatus (or devices such as a CPU or MPU) that reads out and executes a program recorded on a memory device to perform the functions of the above-described embodiments, and by a method, the steps of which are performed by a computer of a system or apparatus by, for example, reading out and executing a program recorded on a memory device to perform the functions of the above-described embodiments. For this purpose, the program is provided to the computer for example via a network or from a recording medium of various types serving as the memory device (e.g., computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-157966 filed on Jul. 13, 2012, which is hereby incorporated by reference herein in its entirety.

What is claimed is:

1. A mobile radiation imaging apparatus comprising:
a generation unit configured to emit radiation;
a positioning unit configured to support the generation unit;
a moveable cart configured to moveably hold the positioning unit;
a storing unit for a radiation imaging unit that includes a radiation sensor and a first battery, the storing unit having a terminal that supplies power to the radiation imaging unit;
a second battery configured to connect to the terminal, the generation unit, and a cable for receiving a supply of power from an external power source; and
a control unit configured to control power supply from the second battery to the first battery based on a remaining amount of the first battery and a remaining amount of the second battery.

2. The radiation imaging apparatus according to claim 1,
wherein the control unit estimates a first number of possible image captures from the remaining amount of the first battery, and a second number of possible image captures from the remaining amount of the second battery, and
if the second number of possible image captures is greater than the first number of possible image captures, the control unit executes power supply from the second battery to the first battery.

3. The radiation imaging apparatus according to claim 1,
wherein the generation unit further comprises a movement detection unit configured to detect whether or not the generation unit is in a moving state, and
the control unit stops power supply from the second battery for driving the generation unit to the generation unit if the movement detection unit detected that the generation unit is in the moving state.

4. The radiation imaging apparatus according to claim 3,
wherein the control unit executes power supply from the second battery for driving the generation unit to the generation unit if the movement detection unit detected that the generation unit is not in the moving state.

5. The radiation imaging apparatus according to claim 4,
wherein the control unit stops power supply from the second battery to the first battery if power supply from the second battery to the generation unit is executed.

6. The radiation imaging apparatus according to claim 1,
wherein the first battery is built into the radiation imaging unit, and
the control unit executes power supply from the second battery to the first battery in a state in which the radiation imaging unit is stored in the storing unit and is connected to a charger that is included in the storing unit.

7. The radiation imaging apparatus according to claim 1,
wherein the first battery is detachable from the radiation imaging unit,
the generation unit further comprises a battery storing unit configured to store the first battery,
and the control unit executes power supply from the second battery to the first battery in a state in which the first battery is stored in the battery storing unit and is connected to a charger that is included in the battery storing unit.

8. The radiation imaging apparatus according to claim 6,
wherein the charger is supported to the storing unit that vibrates in response to movement of the moveable cart via an anti-vibration member such that vibration is prevented.

9. The radiation imaging apparatus according to claim 8, further comprising:
a latch member configured to restrict a relative position of the radiation imaging unit relative to the charger.

10. The radiation imaging apparatus according to claim 1,
wherein a plurality of radiation imaging units are provided,
the radiation imaging units each include a first battery configured to drive the radiation imaging unit, and
the control unit controls power supply from the second battery to each of the first batteries based on a total remaining amount of the first batteries and a remaining amount of the second battery.

11. The radiation imaging apparatus according to claim 10,
wherein the control unit estimates a first number of possible image captures from the total remaining amount of the first batteries, as well as a second number of possible image captures from a remaining amount of the second battery, and
the control unit executes power supply from the second battery to the first batteries if the second number of possible image captures is greater than the first number of possible image captures.

12. The radiation imaging apparatus according to claim 1,
wherein the radiation imaging unit further comprises a display unit configured to display a remaining amount of the first battery.

13. A radiation imaging apparatus having a generation unit that irradiates a subject with radiation, and a radiation imaging unit that captures an image of radiation that passes through the subject,
the generation unit comprising:
a storing unit configured to store the radiation imaging unit or a first battery for driving the radiation imaging unit, and
a second battery configured to drive the generation unit, the second battery being able to supply power to the first battery due to the radiation imaging unit or the first battery being stored in the storing unit and being connected to a charger that is included in the storing unit,
wherein the charger is supported to the storing unit via an anti-vibration member such that vibration is prevented.

14. A mobile radiation imaging apparatus comprising:
a generation unit configured to emit radiation;
a positioning unit configured to support the generation unit;
a moveable cart configured to moveably hold the positioning unit;
a storing unit for a radiation imaging unit that includes a radiation sensor and a first battery, the storing unit having a terminal that supplies power to the radiation unit;
a second battery configured to connect to the terminal, the generation unit, and a cable for receiving a supply of power from an external power source;
a communication circuit configured to receive an imaging order from an external apparatus; and
a control unit configured to control power supply from the second battery to the first battery based on the received imaging order.

15. A mobile radiation imaging apparatus comprising:
a generation unit configured to emit radiation;
a positioning unit configured to support the generation unit;
a moveable cart configured to moveably hold the positioning unit;
a first terminal configured to supply power to a first battery that is detachable from a radiation imaging unit that includes a radiation sensor;
a storing unit for the radiation imaging unit configured to include a second terminal for supplying power to a second battery mounted in the radiation imaging apparatus;
a third battery configured to connect to the first and second terminals, the generation unit, and a cable for receiving a supply of power from an external power source; and
a control unit configured to control power supply from the third battery to the first battery based on a remaining amount of the first, second, and third batteries.

16. A radiation imaging apparatus comprising:
a radiation detection panel configured to detect radiation;
a processing circuit configured to process an electrical signal obtained by the radiation detection panel;
a housing configured to store the radiation detection panel and the processing circuit;
a battery that is detachable from the housing and is configured to supply power to the radiation detection panel and the processing circuit, the battery being provided on a side of the radiation detection panel that is opposite to an incoming radiation surface;
a display unit that is provided on a first side surface of the housing and is configured to display a status of the battery; and
a terminal that is provided on a second surface on a side that is opposite to the first side and is configured to supply power to the battery.
